(19) United States
(12) Patent Application Publication
(10) Pub. No.: US 2007/0186300 A1
(43) Pub. Date: Aug. 9, 2007

(54) Transgenic mouse capable of over-expressing VE-PTP

(76) Inventors: David T. Shima, Barnet (GB); Sofia Ioannidou, Boston, MA (US)

Correspondence Address: (OSI) EYETECH, INC., 41 PINELAWN ROAD, MELVILLE, NY, 11747, US

(21) Appl. No.: 11/271,624

(22) Filed: Nov. 10, 2005

Related U.S. Application Data
(60) Provisional application No. 60/628,084, filed on Nov. 15, 2004.

(51) Int. Cl. A01K 67/027

(52) U.S. Cl. 800/18

(57) ABSTRACT

The present invention relates generally to a transgenic mouse. In particular, the present invention relates to a transgenic mouse whose genome comprises a TetO-VE-PTP transgene. The present invention also relates to a double transgenic mouse whose genome comprises the transgenes TetO-VE-PTP and Tie2-tTA, which is capable of conditionally over-expressing VE-PTP which is contingent on the presence of doxycycline. The present invention also relates to methods of generating a transgenic mouse whose genome comprises a TetO-VE-PTP and a double transgenic mouse whose genome comprises the transgenes TetO-VE-PTP and Tie2-tTA.

### RELATED APPLICATION

This Application claims the benefit of U.S. Provisional Application No. 60/628,084, filed on Nov. 15, 2004. The entire teachings of the above application is incorporated herein by reference.

### FIELD OF THE INVENTION

The present invention relates generally to a transgenic mouse. In particular, the present invention relates to a transgenic mouse whose genome comprises a TetO-VE-PTP transgene. The present invention also relates to a double transgenic mouse whose genome comprises the transgenes TetO-VE-PTP and Tie2-tTA, which is capable of conditionally over-expressing VE-PTP, which is contingent on the presence of doxycycline.

### BACKGROUND OF THE INVENTION

Vascular permeability, similar to blood vessel growth, is thought to be governed by the coordinated and competing actions of Receptor Tyrosine Kinases (RTKs) and Receptor Protein Tyrosine Phosphatases (RPTPs) (Jones, N., et al., Tie receptors: new modulators of angiogenic and lymphangiogenic responses. Nat Rev Mol Cell Biol, 2001, 2(4): p. 257-67; Hanahan, D., Signaling vascular morphogenesis and maintenance. Science, 1997, 277(5322): p. 48-50; Risau, W., Mechanisms of angiogenesis. Nature, 1997. 386(6626): p. 671-4). Extensive experimental evidence has implicated the Vascular Endothelial Growth Factor A (VEGF-A) and Angiopoietin (Ang) signaling cascades in the regulation of permeability, through the activation of the endothelial cell-specific RTKs, VEGFR-2 and Tie-2 respectively. Among the tyrosine phosphatases present in endothelial cells, the recently characterized Vascular Endothelial Protein Tyrosine Phosphatase (VE-PTP) is specifically expressed in this tissue (Fachinger, G., U. Deutsch, and W. Risau, Functional interaction of vascular endothelial-protein-tyrosine phosphatase with the angiopoietin receptor Tie-2. Oncogene, 1999, 18(43): p. 5948-53). A role for VE-PTP in the control of paracellular permeability has been supported by evidence for an interaction with the junctional protein VE-Cadherin (Brady-Kalnay, S. M. and N. K. Tonks, Protein tyrosine phosphatases as adhesion receptors. Curr Opin Cell Biol, 1995. 7(5): p. 650-7). VE-PTP has been shown to reverse VEGF-R2-mediated tyrosine phosphorylation of VE-cadherin, and to increase the integrity of the endothelial monolayer in a cell-based assay. An association between VE-PTP and the seemingly opposing Tie-2 pathway of endothelial barrier regulation has been suggested from co-precipitation experiments, however no functional effect has been shown.

Embryonic lethality in mice with targeted disruption of components in VEGF and Ang signaling cascades has precluded the analysis of the precise physiological function of these pathways during later stages of vascular development and adulthood (Carmeliet, P., et al., Targeted deficiency or cytosolic truncation of the VE-cadherin gene in mice impairs VEGF-mediated endothelial survival and angiogenesis. Cell, 1999. 98(2): p. 147-57362; Dumont, D. J., et al., Dominant-negative and targeted null mutations in the endothelial receptor tyrosine kinase, tek, reveal a critical role in vasculogenesis of the embryo. Genes Dev, 1994. 8(16): p. 1897-909; Carmeliet, P., et al., Abnormal blood vessel development and lethality in embryos lacking a single VEGF allele. Nature, 1996. 380(6573): p. 435-9; Sato, T. N., et al., Distinct roles of the receptor tyrosine kinases Tie-1 and Tie-2 in blood vessel formation. Nature, 1995. 376(6535): p. 70-4; Shalaby, F., et al., Failure of blood-island formation and vasculogenesis in Flk-1-deficient mice. Nature, 1995. 376(6535): p. 62-6; Suri, C., et al., Requisite role of angiopoietin-1, a ligand for the TIE2 receptor, during embryonic angiogenesis. Cell, 1996. 87(7): p. 1171-80).

Conditional gene expression in mice is a popular tool aimed at dissecting the precise roles of genes in complex physiological processes (Lewandoski, M., Conditional control of gene expression in the mouse. Nat Rev Genet, 2001. 2(10): p. 743-55). Among binary over-expression systems based on transcriptional transactivation, the tetracycline-resistance system has seen the widest use in the study of spatially- and temporally-specific gene function (Gossen, M., A. L. Bonin, and H. Bujard, Control of gene activity in higher eukaryotic cells by prokaryotic regulatory elements. Trends Biochem Sci, 1993. 18(12): p. 471-5; Cronin, C. A., W. Gluba, and H. Scrable, The lac operator-repressor system is functional in the mouse. Genes Dev, 2001. 15(12): p. 1506-17; Dor, Y., et al., Conditional switching of VEGF provides new insights into adult neovascularization and pro-angiogenic therapy. Embo J, 2002. 21(8): p. 1939-47; Tremblay, P., et al., Doxycycline control of prion protein transgene expression modulates prion disease in mice. Proc Natl Acad Sci USA, 1998. 95(21): p. 12580-5; Gross, C., et al., Serotonin1A receptor acts during development to establish normal anxiety-like behaviour in the adult. Nature, 2002. 416(6879): p. 396-400; Yang, L. L., et al., Conditional cardiac overexpression of endothelin-1 induces inflammation and dilated cardiomyopathy in mice. Circulation, 2004. 109(2): p. 255-61; Suarez, J., et al., Doxycycline inducible expression of SERCA2a improves calcium handling and reverts cardiac dysfunction in pressure overload-induced cardiac hypertrophy. Am J Physiol Heart Circ Physiol, 2004. 287(5): p. H2164-72; You, X. M., et al., Conditional expression of a dominant-negative c-Myb in vascular smooth muscle cells inhibits arterial remodeling after injury. Circ Res, 2003. 92(3): p. 314-21).

### SUMMARY OF THE INVENTION

Applicants herein describe the generation and characterization of a TetO-VE-PTP responder line transgenic mice, using transgenes that are likely to promote a gain or loss of VE-PTP function. Inducible transgenic lines of full-length and extracellular domain TetO-VE-PTP have been generated.

In one aspect, the present invention relates to a responder line transgenic mouse whose genome comprises a TetO-VE-PTP transgene.

In another aspect, the present invention relates to a double transgenic mouse whose genome comprises the transgenes TetO-VE-PTP and Tie2-tTA. In one embodiment the double transgenic mouse, whose genome comprises the transgenes TetO-VE-PTP and Tie2-tTA, comprises the capability of conditionally over-expressing VE-PTP. In one particular embodiment, the over expression of VE-PTP is contingent on the presence of doxycycline.

In another aspect, the present invention relates to a method of producing a responder line transgenic mouse whose genome comprises a TetO-VE-PTP transgene comprising the steps of:
a) introducing a DNA solution comprising TetO-VE-PTP into mouse oocytes;
b) transferring the mouse oocytes to a pseudo-pregnant surrogate female mouse; and
c) allowing the oocytes to develop to term thereby producing a responder line transgenic mouse whose genome comprises a TetO-VE-PTP transgene.

In one particular embodiment, the responder line transgenic mouse is C57BL/6 or C57BL/6×CBA.

In another aspect, the present invention relates to a method of producing a double transgenic mouse, whose genome comprises the transgenes TetO-VE-PTP and Tie2-tTA. In one embodiment the method comprises the step of crossing a transgenic mouse responder line comprising TetO-VE-PTP with a transgenic mouse driver line comprising Tie2-tTA.

In one particular embodiment, the double transgenic mouse is C57BL/6 or C57BL/6×CBA.

The responder line transgenic mice comprising the transgene TetO-VE-PTP and the double transgenic mice of the present invention have several advantages. For example, transgenic mice comprising the transgenes TetO-VE-PTP and Tie2-tTA may be used to analyze the role of VE-PTP in regulating paracellular permeability in vivo. The transgenic mice of the present invention may also be used as a model of VE-PTP dysfunction. Combined with a driver line that restricts expression to the vasculature, the transgenic mice constitute tools to manipulate VE-PTP function and probe its role within the context of endothelial barrier assembly, maintenance, and breakdown.

### BRIEF DESCRIPTION OF THE DRAWINGS

The file of this patent contains at least one drawing executed in color. Copies of this patent with color drawings will be provided by the Patent and Trademark Office upon request and payment of the necessary fee.

FIG. 1 is a schematic showing the tetracycline-responsive regulatory system for transcriptional transactivation. (A) The tetracycline transactivator of transcription (tTA) is encoded by a chimeric construct of the E. coli Tn10tetR gene (bottom) and the VP16 transactivation domain (top). In the absence of Doxycycline (Dox), tTA dimers bind to the tetracycline operon, consisting of seven tandemly repeated 19-bp tetO sequences (tetO7), and activate transcription of the gene of interest (target ORF) from a minimal promoter (TATA). When bound to Dox, tTA undergoes a conformational change that prevents it from binding tetO7. (B) In the reverse tTA (rtTA) system, the tetR gene has been mutated so that tTA binds to tetO7 only in the presence of Dox. Only upon binding of Dox, does tTA undergo the necessary conformational change that allows it to bind to tetO7 to activate transcription. (ORF, open reading frame; pA, polyadenylation site; TSP, tissue specific promoter). (Reproduced from Maegawa, H., et al., Expression of a dominant negative SHP-2 in transgenic mice induces insulin resistance. J Biol Chem, 1999. 274(42): p. 30236-43)

FIG. 2 is a schematic showing the dox-repressible binary transgenic system. (A) Schematic of driver and responder transgenes. Responder transgenes for VE-PTP full-length (FL), extracellular domain only (EC), and trapping mutant (R/A) are depicted. The black region represents a chimaeric intron. Arrows denote PCR primers. (B) Genotyping results for a wildtype (a) and a responder transgenic (b) mouse, using a dual PCR to amplify the transgene (340 bp), and the endogenous (200 bp) copies. The two amplifications share the same reverse primer, but differ in their forward primers. The transgene-specific primer is shown in (A), while the endogenous-specific primer anneals in a 5′untranslated region present only in the endogenous copy.

FIG. 3 shows VE-PTP induction in vitro. MEF 3T3 cells expressing the tTA transactivator were transfected with plasmids containing CMV-VE-PTP (A) TetO-VE-PTP (B,D), and TetO-VE-PTP R/A (C). Doxycycline (2 μg/ml) was included in the media of cells in (D) to suppress the tTA-driven transcription. Immunolabeling for VE-PTP (green), and the golgi marker GM130 (red) showed that VE-PTP is constituvely expressed in (A), induced in (B) and (C), and repressed in (D). Optical sections were taken at the level of the golgi. (Bar=20 μm)

FIG. 4 show images of VE-PTP induction ex vivo. Mouse embryonic fibroblasts (MEFs) isolated from responder TetO-VE-PTP founder lines were transfected with a plasmid expressing the reverse tetracycline transactivator (rtA) (A,B), and cultured in the absence (A) or in the presence (B) of doxycycline (2 μg/ml). Immunolabeling for VE-PTP (red channel) and GM 130 (green channel) demonstrated that VE-PTP is expressed only when doxycycline is included in the medium (A,B). MEFs co-transfected with the tTA plasmid plus the plasmid used in the generation of the TetO-VE-PTP lines, and cultured with doxycycline, were used as a positive control (C). Optical sections were captured at the level of the golgi (A-C), or the plasma membrane (D). VE-PTP labeling throughout the plasma membrane is observed, with an enrichment in peripheral areas.

FIG. 5 is a schematic showing a method for VE-PTP transcript analysis in vivo. Tie2-tTA drivers are confirmed as capable of driving transcription of a LacZ reporter gene in the endothelium at E11.5 (A). Schematic of Real-time PCR primer and probe set positions (B). Primers are denoted by top or bottom arrows, and probes by grey lines. Probes and reverse primers are common for endogenous and transgene VE-PTP transcripts, but forward primers (top arrows) are unique to the particular version. RT-PCR primers for transgene VE-PTP were designed in the same region as for Real-time PCR. For simplicity, only one version of the three transgene constructs is shown, and introns are omitted from the endogenous copy.

FIG. 6 shows electrophoresis gels representing screening through TetO-VE-PTP founder lines by RT-PCR. Screening through Tet-VE-PTP founder lines by RT-PCR. TetO-VE-PTP and p97 transcripts were amplified in the same reaction, from RNA that was reverse transcribed with the inclusion (+) or the omission (−) of reverse transcriptase. A 153 bp fragment was amplified in samples containing the Tet-VE-PTP transcript, while a 470 bp fragment from the constitutively expressed p97 gene was amplified in all samples. The presence of a 153 bp band for Tet-VE-PTP or a 670 bp band for p97 (after inclusion of an intron) in the (−) lane was indicative of a contribution by genomic DNA. The founder lines and genotypes are indicated on top of each gel picture. The nomenclature used for each founder line consists of a four-digit number plus one or two letters, collectively indicating each event of oocyte transfer into a pseudopregnant female. Numbers following letters in the nomenclature distinguish different founders stemming from the same oocyte transfer event. The genotype of the animals are abbreviated using the following scheme: t=Tie2-tTA transgene, v=Tet-VE-PTP transgene. Accordingly, double transgenic animals are denoted as t/v, while single transgenic animals for the driver transgene or the responder transgene are denoted as t/- and -/v, respectively. Molecular weight markers and the identity of the obtained bands are indicated.

FIG. 7 TetO-VE-PTP transcript levels in representative E11.5 embryos from different founder lines were measured in a single Taqman experiment. Transcript levels are expressed as a fold induction over a sample with very low amounts of transcript, namely 3530B t/v. (A) differs from (B) in lacking the samples with the highest 6 scores, to highlight differences at low transcript levels. The genotypes and founder lines of the animals are denoted beneath the graph, according to the scheme described in FIG. 6.

FIG. 8 is a chart showing normalized TetO-VE-PTP transgene induction in double over single transgenic littermates. Transcript levels for transgene and endogenous VE-PTP in each E11.5 embryo were measured in the same TaqMan experiment. For each sample, the transgene transcript levels were normalized against the endogenous VE-PTP transcript levels. The fold induction for normalized TetO-VE-PTP transcript levels in double transgenic embryos versus their single transgenic littermates is plotted for selected founder lines. A fold induction value of 1 indicates no difference between the normalized levels of littermates. Founder lines along with the TetO-VE-PTP constructs they harbour are denoted.

FIG. 9 is a chart showing Tie2-tTA transcript levels in representative E11.5 embryos from different founder lines were measured in a single Taqman experiment. Transcript levels were expressed as a fold induction over sample 3530B. The genotypes and founder lines of the animals are denoted beneath the graph according to the scheme described in FIG. 6.

FIG. 10 shows images of abnormalities in double transgenic embryos observed in two different litters (A and B) of founder line 3597AB. E11.5 double transgenic (t/v) and single transgenic littermates from crosses between Tie2-tTA and Tet-VE-PTP EC mice, of responder line 3597AB. The double transgenic embryos in both litters were developmentally retarded in comparison to their littermates, displayed hemorrhages (arrowhead) and enlarged pericardia (arrows). The partially dissected yolksac of the double transgenic embryo in (A) is marked by an asterisk.

### DETAILED DESCRIPTION OF THE INVENTION

In one aspect, the present invention relates to a responder line transgenic mouse whose genome comprises a TetO-VE-PTP transgene.

In another aspect, the present invention relates to a double transgenic mouse whose genome comprises the transgenes TetO-VE-PTP and Tie2-tTA. In one embodiment the double transgenic mouse, whose genome comprises the transgenes TetO-VE-PTP and Tie2-tTA, comprises the capability of conditionally over-expressing VE-PTP. In one particular embodiment the over expression of VE-PTP is contingent on the presence of doxycycline.

In another aspect, the present invention relates to a method of producing a responder line transgenic mouse whose genome comprises a TetO-VE-PTP transgene comprising the steps of:
a) introducing a DNA solution comprising TetO-VE-PTP into mouse oocytes;
b) transferring the mouse oocytes to a pseudo-pregnant surrogate female mouse; and
c) allowing the oocytes to develop to term thereby producing a responder line transgenic mouse whose genome comprises a TetO-VE-PTP transgene.

In one particular embodiment, the responder line transgenic mouse is C57BL/6 or C57BL/6×CBA.

In another aspect, the present invention relates to a method of producing a double transgenic mouse, whose genome comprises the transgenes TetO-VE-PTP and Tie2-tTA. In one embodiment the method comprises the step of crossing a transgenic mouse responder line comprising TetO-VE-PTP with a transgenic mouse driver line comprising Tie2-tTA.

In one particular embodiment, the double transgenic mouse is C57BL/6 or C57BL/6×CBA.

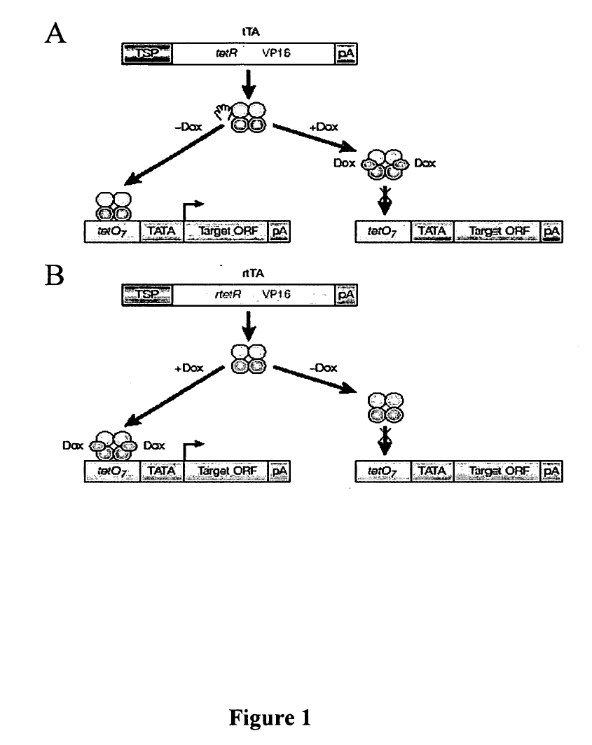

Applicants describe herein a conditional over-expression system to perturb the function of VE-PTP in a tightly controlled spatial and temporal manner. Such qualities are fulfilled by the tetracycline-responsive binary expression system, which is based on the E. coli tetracycline resistance operon (FIG. 1) (Gossen, M. and H. Bujard, Tight control of gene expression in mammalian cells by tetracycline-responsive promoters. Proc Natl Acad Sci USA, 1992. 89(12): p. 5547-5; 1Furth, P. A., et al., Temporal control of gene expression in transgenic mice by a tetracycline-responsive promoter, Proc Natl Acad Sci USA, 1994. 91(20): p. 9302-6; Gossen, M., et al., Transcriptional activation by tetracyclines in mammalian cells. Science, 1995. 268(5218): p. 1766-9; Kistner, A., et al., Doxycycline-mediated quantitative and tissue-specific control of gene expression in transgenic mice. Proc Natl Acad Sci USA, 1996. 93(20): p. 10933-8). The system relies on two parts; a driver transgene harbouring a recombinant tetracycline transactivator (tTA) and a responder transgene, harbouring the gene of interest under the control of the tetracycline operon (tetO). When a driver line of mice is crossed to a responder line of mice, induction of expression for the gene of interest is achieved as a result of the high affinity of tTA for the tetO element. Spatial control of gene expression is ensured by placing the tTA downstream of tissue-specific promoters (Mayford, M., et al., Control of memory formation through regulated expression of a CaMKII transgene. Science, 1996. 274(5293): p. 1678-83; Sarao, R. and D. J. Dumont, Conditional transgene expression in endothelial cells. Transgenic Res, 1998. 7(6): p. 421-7; Yu, Z., C. S. Redfern, and G. I. Fishman, Conditional transgene expression in the heart. Circ Res, 1996. 79(4): p. 691-7; Jaubert, J., et al., Tetracycline-regulated transactivators driven by the involucrin promoter to achieve epidermal conditional gene expression. J Invest Dermatol, 2004. 123(2): p. 313-8; Ju, H., et al., Conditional and targeted overexpression of vascular chymase causes hypertension in transgenic mice. Proc Natl Acad Sci USA, 2001. 98(13): p. 7469-74; Manickan, E., et al., Conditional liver-specific expression of simian virus 40 T antigen leads to regulatable development of hepatic neoplasm in transgenic mice. J Biol Chem, 2001. 276(17): p. 13989-94). In one embodiment, temporal control of gene expression is achieved through the use of the antibiotic doxycycline, which can reversibly suppress the binding of tTA to the tetO element, thereby preventing induction of the gene of interest.

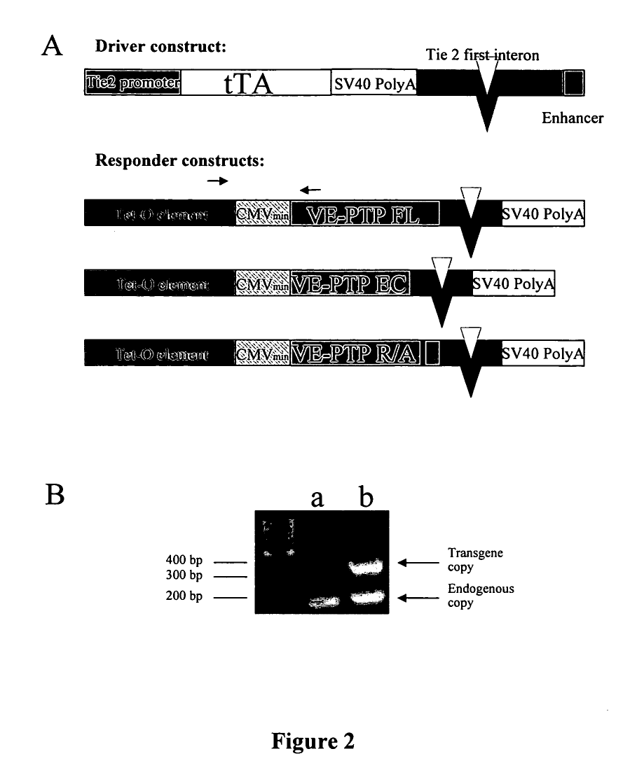

Responder line constructs were designed to produce wildtype or dominant negative versions of the VE-PTP protein. In one embodiment, three separate versions of the VE-PTP cDNA were used: 1) wildtype, full-length cDNA, 2) VE-PTP extracellular (EC) domain mutant, and 3) VE-PTP catalytic domain trapping mutant (R/A). Given that VE-PTP and VE-Cadherin interact via the 17^{th} membrane proximal FNIII domain and the 5^{th} membrane proximal cadherin domain respectively, induced VE-PTP EC is expected to interfere with the extracellular domain of the substrates and possibly obstruct binding of the wildtype phosphatase. The R/A trapping mutant binds to the substrate and, whilst unable to perform catalysis, does not release it, potentially preventing the recruitment of the wildtype phosphatase or other signaling molecules downstream. cDNAs were introduced into an expression vector, downstream of a tetracycline response element linked to a CMV minimal promoter, and upstream of an artificial intron, and a polyadenylation signal (FIG. 2A).

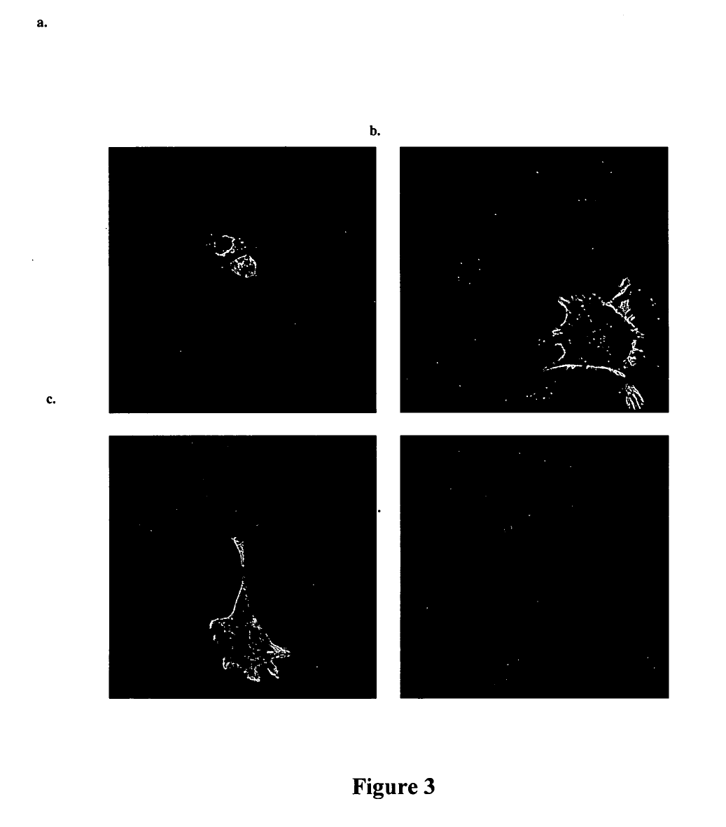

A prerequisite for injections is ensuring that the constructs expressed VE-PTP protein. A commercially available MEF 3T3 TetO-Off cell line was transfected with the constructs for VE-PTP full-length, and VE-PTP R/A, and expression of the protein was assessed by immunofluorescence using a rabbit anti-serum (FIG. 3). A plasmid harbouring CMV-driven full-length VE-PTP was used as a positive control for VE-PTP expression, and the antibiotic doxycycline was omitted or included in the culture medium to suppress the tTA-driven induction of VE-PTP. VE-PTP was induced in the absence of doxycycline from both constructs (FIG. 3 B,C), and its expression was suppressed when doxycycline was included in the culture medium. Cell death was observed among the population of transfected cells, consistent with previous observations during VE-PTP overexpression in CHO or COS cells. The surviving transfected cells expressed VE-PTP on their plasma membrane, but had altered morphology and sometimes appeared retracted from the substratum.

Generation of TetO-VE-PTP responder mice is herein disclosed. The TetO-VE-PTP transgenes were excised from their respective plasmids and injected in the pronuclei of mice from two different backgrounds. In one embodiment, the mice are C57BL6/J or B6CBAF1×F1 hybrid mice (Table I). ‘Responder line’ offspring were obtained for all transgenes, as assessed by PCR (FIG. 2B), and all but one of the identified founder animals transmitted the transgene to their progeny. The total of 16 founder animals that were capable of transmitting the transgene, once bred to C57BL6/J or B6CBAF1×F1 hybrid mice produced normal litter sizes of about 8 pups. Overall, the presence of the responder transgene alone did not appear to impart negatively on mouse development, and the problems encountered within founder line 3650B2 are suggestive of a disruption of a normal function caused by transgene insertion.

TABLE I
SUMMARY OF DNA INJECTION RESULTS

| | | Born/ | | Trans- |
|---|---|---|---|---|
| | | Trans- | | genic/ |
| Construct | Strain | ferred % | Transgenic | Born % |
| Tet VE-PTP | C57BL6/J | 10% | 4 | 12% |
| Tet VE-PTP | B6CBAF1 × F1 | 11% | 3 | 15% |
| Tet VE-PTP EC | B6CBAF1 × F1 | 9.8% | 4 | 14.8% |
| Tet VE-PTP R/A | B6CBAF1 × F1 | 20% | 5 | 8.9% |

Table I is a summary of DNA injection records per transgene construct in a particular mouse background. ‘Born/Transferred’ stands for the total number of offspring born as a percentage of the number of injected oocytes that were transferred to pseudopregnant mothers. The percentage of transgenic animals within this offspring is represented by ‘Transgenic/Born’.

Screening Responderfounder Lines for VE-PTP Expression

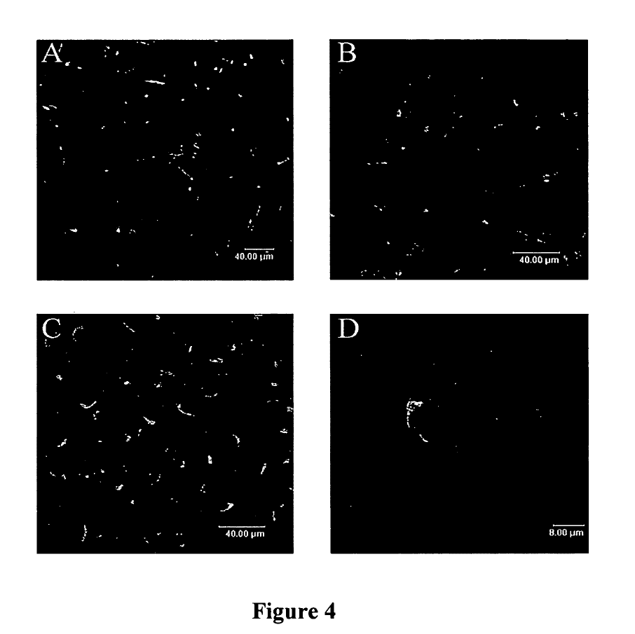

Ex vivo inducibility was screened. The first attempt to screen between the different founder lines was based on selecting for VE-PTP inducibility without breeding to the driver lines. Mouse embryonic fibroblasts (MEFs) are isolated from E14.5 mice and transfected with an rtTA expressing construct (pTetOn). rtTA, the reverse tetracycline transactivator, differs from tTA by only four amino acids, and drives expression of the target gene upon addition of doxycycline to the medium (FIG. 3A). As a positive control, MEFs were co-transfected with the plasmid containing the TetO-VE-PTP transgene in addition to the rtTA plasmid. Immunofluorescence analysis using rabbit anti-VE-PTP antiserum revealed that stably integrated transgenes could be induced to express VE-PTP ex vivo in the presence of an exogenous transactivator element (FIG. 4B). Moreover this expression was contingent upon the presence of doxycycline (FIG. 4B), as no expression was observed in the absence of the antibiotic (FIG. 4A). Co-transfected cells exhibited higher levels of protein expression, due to the presence of multiple copies of the transgene within a plasmid versus when it is stably integrated in the genome (FIG. 4 C, D).

Screening for Induction in Double Transgenic Embryos

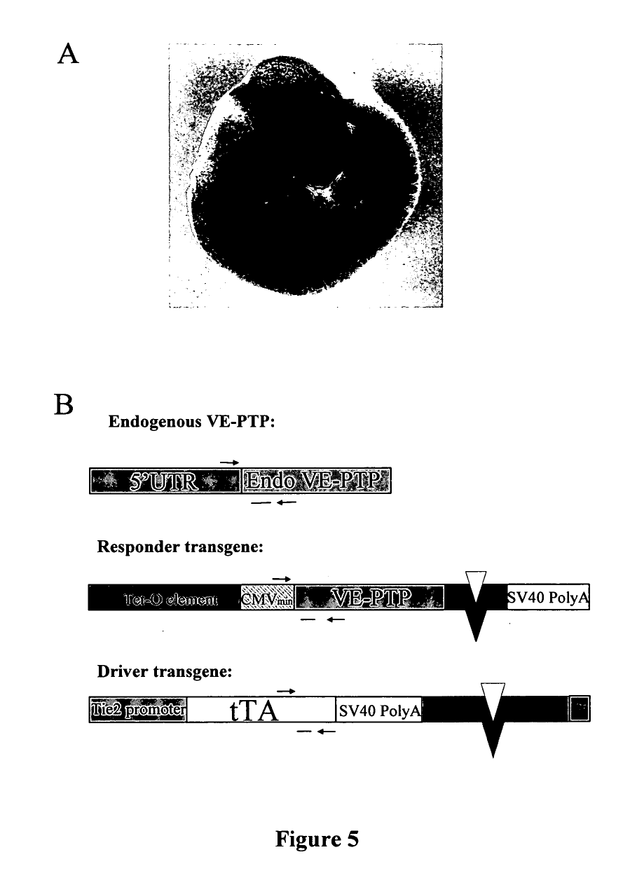

Screening for induction in vivo was selected. This method assessed the capability of transgene induction from a given integration site within the context of relevant levels of the tTA driver in the organism. Using an RNA-based screening method, the induction of the soluble VE-PTP EC can be monitored, in contrast to antibody-based approaches. A Tie2-tTa driver line of mice (provided by Dr. Urban Deutsch; Max-Planck Institute, Bad Nauheim, Germany), capable of driving uniform vascular endothelial expression in embryos was crossed to each of the responder founder lines, and embryos were harvested at E11.5 (Schlaeger, T. M., et al., Uniform vascular-endothelial-cell-specific gene expression in both embryonic and adult transgenic mice. Proc Natl Acad Sci USA, 1997. 94(7): p. 3058-63 Schlaeger, T. M., et al., Uniform vascular-endothelial-cell-specific gene expression in both embryonic and adult transgenic mice. Proc Natl Acad Sci USA, 1997. 94(7): p. 3058-63). During this embryonic stage the Tie2 promoter is thought to be highly active (Dumont, D. J., et al., Vascularization of the mouse embryo: a study of flk-1, tek, tie, and vascular endothelial growth factor expression during development. Dev Dyn, 1995. 203(1): p. 80-92), and Lac Z staining of E11.5 embryos which were double transgenic for the Tie2-tTA transgene and a TetO-Lac Z responder transgene exhibited strong Lac Z expression throughout the vasculature (FIG. 5A).

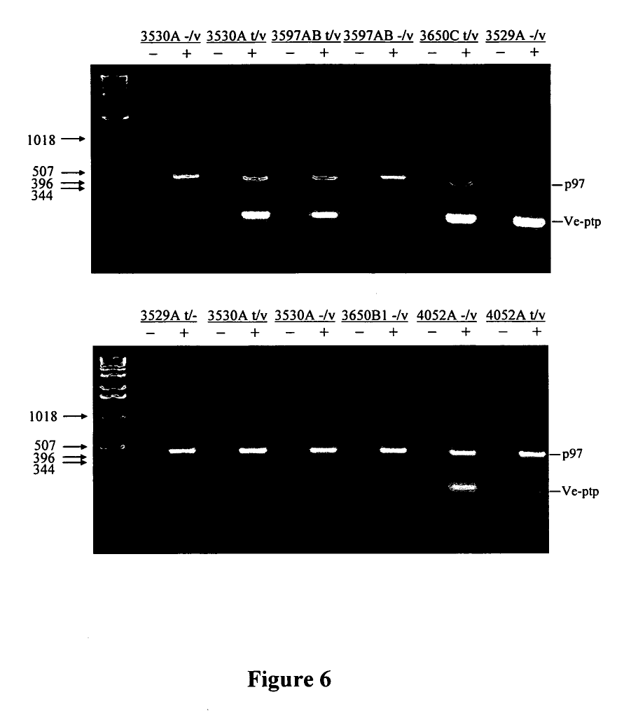

Double transgenic embryos from crosses between the Tie2-tTA line and each of the TetO-VE-PTP responder lines were assessed for the presence of TetO-VE-PTP transcript by RT-PCR. In order to distinguish the transgene version of VE-PTP from the endogenous version, primers were designed to amplify a region unique to the transgene, located at the 5′end (FIG. 5B). A founder line capable of VE-PTP induction is one where the levels of VE-PTP transcript in double transgenic embryos exceeded those in embryos that were single transgenic for the responder transgene (FIG. 6). Some leakiness in single transgenic embryos was evident, while genomic DNA was sometimes observed to contribute to the signal. Given that the 5′end of the TetO-VE-PTP transgene RNA did not differ from the DNA that encoded it, a number of controls were included to ensure that the observed signal was attributable to the presence of a transcript. An RT-PCR reaction using material from a cDNA synthesis without reverse transcriptase was run in parallel to each reaction carried out with cDNA synthesized in the presence of the enzyme. In addition, two transcripts were amplified in the same RT-PCR reaction: p97 and TetO-VE-PTP. The primers for p97 spanned an intron, giving rise to a PCR product of higher molecular weight when DNA, versus RNA, was amplified. When genomic DNA was present, the difference in intensity between the products of the reactions containing or lacking the enzyme was taken as a measure of the RNA levels.

To confirm and also obtain a quantitative estimate of the observations from RT-PCR, the analysis was validated by Real-time quantitative PCR (TaqMan®). This method ensures increased accuracy, as two primers and one probe must anneal to each transcript. This method also adds quantitative value, as product levels are assessed in the linear phase of the reaction, prior to the saturated end-point. Analogously, the RT-PCR screening, a primer and probe set was designed to amplify a region unique to the transgene version of VE-PTP (FIG. 5B). In addition a primer and probe set specific for the endogenous version of VE-PTP was designed, and also a set specific for the Tie2-tTA transcript was designed, to confirm for tTA expression in animals with the appropriate genotype (FIG. 5B).

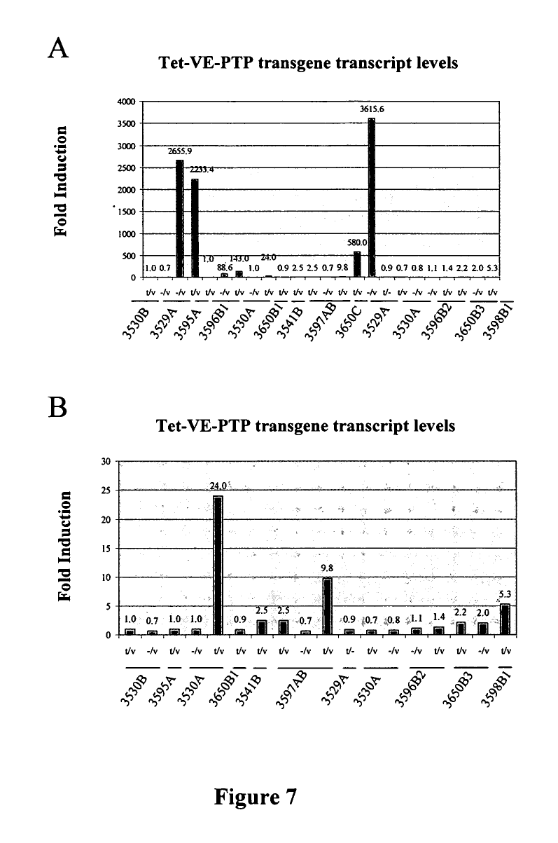

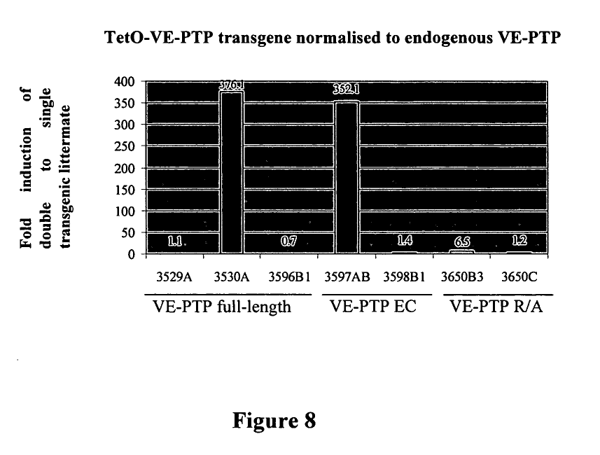

In a first round of analysis, all founder lines were analysed for the induction of VE-PTP, and lines with no or insignificant amplification of the TetO-VE-PTP transgene (namely 3598C, 4052A, and 3664B) were eliminated. Subsequently, a second round of analysis was carried out, where representative samples from all remaining founder lines were compared side by side, in the same Taqman run (FIG. 7). For each founder line, a double transgenic embryo was assessed, and where possible its TetO-VE-PTP single transgenic littermate was included for comparison. This allowed to rank the founder lines according to the transcript levels produced in each one, and also to assess how much of the observed transcript was due to leakiness, even in the absence of the driver, and how much was due to an induction by tTA. To resolve ambiguities within some lines, a follow-up analysis was performed (FIG. 8). The founder lines harboring the highest levels of VE-PTP transcript (3529A and 3650C), were not inducible, as samples displayed similar levels in the presence or the absence of the driver (FIGS. 7A and 8A).

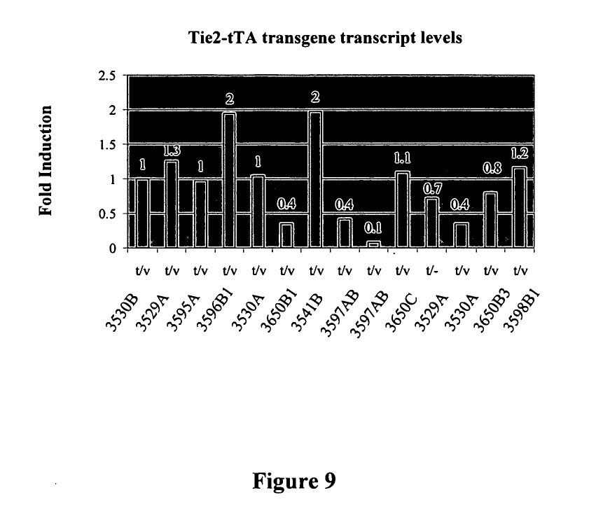

Founder lines with significant levels of inducible (or else, not leaky) VE-PTP were selected for each of the three types of TetO-VE-PTP transgene, to undergo a third-round of analysis (FIG. 9). Given that an over-expression approach depends on flooding the system with a protein that will enhance or override the normal cellular counterpart, an attempt to predict in which founder line it would be more likely for the transgene to have an impact was initiated by measuring the levels of endogenous VE-PTP transcript. Levels of transgene VE-PTP transcript were subsequently normalized to the corresponding levels of the endogenous transgene, and compared between littermates. A high transgene:endogenous ratio in the double transgenic versus the single transgenic sample, as seen in lines 3530A, 3597AB, and 3650B3, was considered as a positive indication (FIG. 9). The non-regulatable founder lines 3529A and 3650C displayed no difference in the transgene:endogenous ratios between littermates. Collectively the above observations (summarized in Table II) suggested that founder lines 3530A, 3597AB, and 3650B3, for TetO-VE-PTP full length, EC only, and R/A mutant, respectively, were useful.

TABLE II

| | | | comparison within founder lines: | | |
|---|---|---|---|---|---|
| | | Rank according | | transgene:endogenous | |
| | | to transgene | | ratio | Phenotype |
| Tet-VE-PTP | | transcript levels | leakiness | upregulation in | Genotype |
| construct | Founder line | (TaqMan) | in —/v | t/v vs —/v | correlation |
| full length | 3529A | 1st | YES | NO | NO |
| | 3530A | 4th | SLIGHT | 376x | partly |
| | 3530B | 10th | NO | N.D. | NO |
| | 3541B | 8th | YES | N.D. | NO |
| | 3595A | 11th | NO | N.D. | NO |
| | 3596B1 | 3rd | YES | NO | NO |
| | 3596B2 | 6th | YES | N.D. | NO |
| EC only | 3597AB | 5th | NO | 352x | partly |
| | 3598B1 | 7th | N.D. | 1.4x | NO |
| | 3598C | — | — | — | YES |
| | 4052A | — | — | — | NO |
| R/A mutant | 3650B1 | 12th | NO | N.D. | NO |
| | 3650B3 | 9th | YES | 6.5x | NO |
| | 3650C | 2nd | YES | NO | NO |
| | 3664B | — | — | — | NO |

Table II Summary of Real-time PCR and morphological observations for TetO-VE-PTP founder lines. Founder lines are ranked according to their transgene transcript levels measured in a parallel comparison within a single TaqMan experiment. Uncontrolled expression in -/v refers to transgene transcription in a single transgenic animal for the responder transgene, in the absence of the tTA transactivator. Normalized transgene induction in double transgenic (t/v) versus their single transgenic (-/v) littermate refers to an empirical estimate of the increase in transgene levels, described in FIG. 8. Phenotypic trends observed in E11.5 embryos are indicated for the appropriate founder lines. The nomenclature for founder lines and genotypes follows the scheme described in FIG. 6. N.D. stands for not determined.

In order to validate the induction at the protein level, double transgenic embryos from inducible and non-regulatable lines were processed for western blotting and wholemount immunostaining with a monoclonal anti-VE-PTP antibody.

Abnormalities in Double Transgenic Embryos

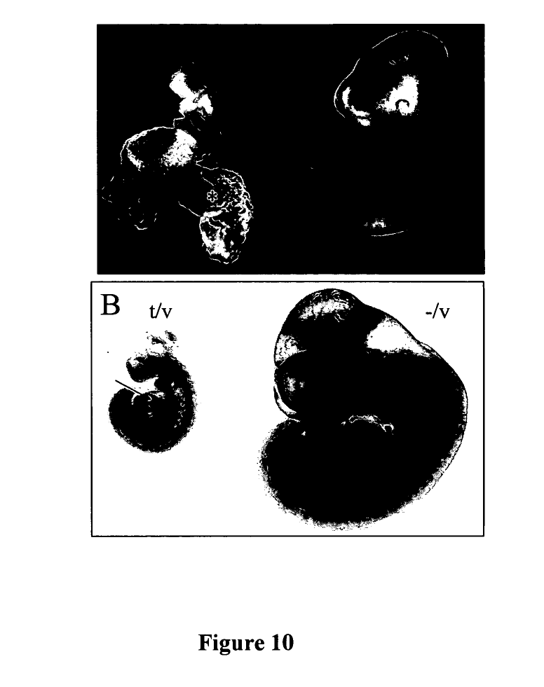

In the course of collecting samples for the transcriptional analysis of VE-PTP induction, a number of abnormalities were encountered in embryos. In some cases, gross morphological abnormalities correlated with a double transgenic genotype (Table II). It should be noted that not all double transgenic animals in these lines exhibited an abnormality. In the more extensively studied 3597AB line, it was evident that within the subset of double transgenic animals, abnormalities correlated with the highest TetO-VE-PTP transcript levels in the litter. Such 3597AB double transgenic embryos were significantly smaller than their littermates, and often exhibited an expanded pericardium with hemorrhages (FIG. 10). Indirect support for an effect of VE-PTP induction on mouse development stems from a preliminary analysis of the genotype statistics in the progeny of 3530A and 3597AB mice crossed to the Tie2-tTA line. A sub-mendelian ratio of double positive animals born from either TetO-VE-PTP founder lines was observed, more so in case of 3597AB (Table III).

TABLE III
Table III. Genotype records for crosses between the Tie2-tTA driver
line and the 3530A or 3597AB responder lines. 10.5-11.5 old embryos,
and 7-day old pups were genotyped for the presence of both transgenes.
The percentage of double transgenic animals in a litter, and the total
number of animals assessed (n) are indicated.

| Founder line | E10.5 | E11.5 | P7 |
|---|---|---|---|
| 3530A | 11.1% (n = 9) | 17.9% (n = 28) | 11.1% (n = 18) |
| 3597AB33% | (n = 6) | 20% (n = 25) | 7.7% (n = 13) |

Table III. Genotype records for crosses between the Tie2-tTA driver line and the 3530A or 3597AB responder lines. 10.5-11.5 old embryos, and 7-day old pups were genotyped for the presence of both transgenes. The percentage of double transgenic animals in a litter, and the total number of animals assessed (n) are indicated.

A conditional over-expression system in the analysis of the function of VE-PTP may have several advantages. First, it should allow one to specifically focus on particular stages in vascular development or disease, sparing the remainder of an organism's physiology from adverse effects. Also, an over-expression approach leading to a loss of function, through the action of a dominant negative version of the protein, should preclude the compensatory mechanisms associated with genetic ablation studies.

In one embodiment, the ideal responder line should be silent for the transgene it harbours, until crossed to the driver line, whereupon it should sustain high levels of expression. Given that inducible lines are often a minority among responder lines (Corbel, S. Y. and F. M. Rossi, Latest developments and in vivo use of the Tet system: ex vivo and in vivo delivery of tetracycline-regulated genes. Curr Opin Biotechnol, 2002. 13(5): p. 448-52), a number of oocytes are typically injected, to give rise to a number of potential founder lines. A total of 17 animals harboring TetO-VE-PTP transgenes were born, out of which 16 were able to transmit the transgene to the next generation. Mice from all but one founder line appeared normal and were reproduced with the expected frequency, suggesting that there were no toxic effects associated with the presence of the transgene. After validating the ability of the responder constructs to support the induction of VE-PTP as plasmids in vitro, and after chromosomal integration ex vivo, the entire complement of responder lines were assessed for induction in vivo by RT-PCR and TaqMan. The different responses observed can be classified in three broad categories of responder mice: 1) Founder lines that did not express TetO-VE-PTP, 2) Founder lines that were non-regulatable, and expressed TetO-VE-PTP even in the absence of the transactivator, and 3) Founder lines that expressed no or very little TetO-VE-PTP in the absence of the transactivator, but could support an induction in its presence.

A critical determinant in the ability of a promoter-less transgene to remain silent, and only be induced in the presence of a transactivator, is the site of chromosomal integration (Robertson, A., et al., Effects of mouse strain, position of integration and tetracycline analogue on the tetracycline conditional system in transgenic mice. Gene, 2002. 282(1-2): p. 65-74). Transgenes can integrate near promoters that lead to their constitutive transcription; the ‘leakiness’ in the absence of the transactivator. On the other hand, integration sites can be surrounded by silencer elements that preclude transcription, even in the presence of the transactivator. Stimulatory or suppressive signals in the genome have also been attributed to factors affecting the three-dimensional structure of chromatin, such as acetylation or methylation (Pikaart, M. J., F. Recillas-Targa, and G. Felsenfeld, Loss of transcriptional activity of a transgene is accompanied by DNA methylation and histone deacetylation and is prevented by insulators. Genes Dev, 1998. 12(18): p. 2852-62). Non-regulatable expression, or absence of expression altogether have been previously reported for responder. The two non-regulatable VE-PTP responder lines (3529A, 3650C) displayed the highest levels of TetO-VE-PTP transcript among founder lines, without however exhibiting any obvious morphological or behavioural defect. If VE-PTP only interacts with particular targets residing in the endothelium, then its ectopic expression should not lead to an adverse phenotype. Another explanation could be the inability of the particular transcripts to be translated into protein. Immunohistochemistry of wholemount embryos and western blotting of embryo lysates, using a monoclonal antibody against VE-PTP, failed to reveal any VE-PTP expression at the protein level.

The two founder lines that displayed the best induction properties (3530A, 3597AB), also exhibited the highest transgene:endogenous ratio, an arbitrary measurement devised to predict the effectiveness of a transgene. Normalizing the relative transgene levels to those of endogenous VE-PTP was deemed more relevant than measuring total (transgene and endogenous) levels, as the latter could be misleading in the case of high endogenous levels in a non-transgenic animal. In addition to this ratio, a number of abnormalities were apparent upon dissection, which could potentially be the consequences of VE-PTP function perturbation. Phenotype to genotype correlations were more consistent in 3597AB animals, a responder line designed to express only the extracellular domain of VE-PTP, and a putative dominant negative of the protein. Moreover, abnormalities in this line could be correlated to the transcript levels that were measured by Real-time PCR. The embryonic phenotypes were also reflected in a preliminary analysis of the progeny from founder lines 3530A and 3597AB, where sub-mendelian ratios were observed for double transgenic animals in a given litter. The few surviving animals would be expected to have low transcript levels, either as a result of incomplete penetrance of the phenotype or due to the existence and positive selection of compensatory mechanisms. Variations in the expression of responder transgenes, even between littermates, had been previously noted. However, in contrast to reports linking responder expression variation to the levels of the transactivator (Bogeroger, H. and P. Gruss, Functional determinants for the tetracycline-dependent transactivator tTA in transgenic mouse embryos. Mech Dev, 1999. 83(1-2): p. 141-53), no correlation was observed among the tTA and TetO-VE-PTP transcript levels among littermates with identical genotype (FIGS. 6 and 7).

Assuming protein translation is carried out effectively, whether attained levels of exogenous VE-PTP protein are sufficient to hamper with its physiological role is a different question. Preliminary observations of vascular defects in double transgenic embryos of line 3597AB are encouraging. Embryos were developmentally delayed, had enlarged pericardia, and hemorrhages in the heart and brain areas. Heart phenotypes are a common theme in many mutants that perturb the function of components of the vasculature.

### EXAMPLES

The following examples serve to illustrate certain useful embodiments and aspects of the present invention and are not to be construed as limiting the scope thereof. Alternative materials and methods can be utilized to obtain similar results.

All chemicals were purchased from Sigma-Aldrich, and Fluka, unless otherwise indicated. Phosphate buffer Saline without calcium or magnesium (PBS), LB medium, LB-agar, EDTA, trypsin/versene, glutamine, penicillin/streptomycin, Leibovitz L-15 medium were provided by CRUK or Eyetech Research Center central services.

### Example 1

Electrocompetent Bacteria

Preparation of Electrocompetent Cells

5 ml from an overnight culture of XL-1 blue bacteria (Stratagene), were added to 800 ml of LB medium and grown at 37° C. to an OD₅₉₅ of 0.6. The culture was incubated for 30 minutes on ice and centrifuged at 5000 rpm for 20 minutes at 4° C. using a JA-10 rotor of an Avanti J-E centrifuge (Beckman Coulter). The pellet was washed twice with 500 ml ice-cold water, once with 40 ml ice-cold 10% glycerol, and was finally resuspended in 1 volume ice-cold 10% glycerol. Aliquots of the preparation were stored at −80° C.

Transformation

50-500 pg DNA were added to 80 μl of electrocompetent cells in a 1 mm electroporation cuvette (Bio-Rad). Mixtures were incubated on ice for 10 minutes and transformations were performed at 200%, 25 μF, and 1.8 kV. Bacteria were incubated with 400 μl SOC medium at 37° C. for 1 hour, and centrifuged at full speed in a microcentrifuge. Pellets were resuspended in 100 μl LB medium, plated on agar plates containing Ampicillin (75 μg/ml) and incubated overnight at 37° C.

### Example 2

Chemically Competent Bacteria

Transformation

50-500 pg DNA were added 50 μl of One Shot TOP10 chemically competent E. coli (Invitrogen), incubated on ice for 5 minutes, heat-shocked at 42° C. for 30 seconds, and transferred back to ice. Bacteria were supplemented with 250 μl of SOC medium and grown at 37° C. for 1 hour. Cultures were plated on agar plates containing Ampicillin (75 μg/ml) and incubated overnight at 37° C.

Cryopreservation

Bacterial Cultures were Stored as 50% Glycerol Stocks at −80° C.

### Example 3

Suppression of TetO-Off Constructs by Doxycycline

MEF 3T3 TetO-Off cells transfected with TetO-VE-PTP responder constructs, or MEFs, from transgenic animals, transfected with pTetOn (Clonetech), were incubated in medium containing 2 μg/ml Doxycycline (Sigma) in order to suppress the expression of the VE-PTP transgene.

### Example 4

Preparation of Genomic DNA

Mammalian tissues were digested in 0.5 ml of lysis buffer (100 mM Tris HCl, pH 8.5, 5 mM EDTA, 0.2% SDS, 200 mM NaCl, 0.5 mg/ml proteinase K), shaking at 55° C. overnight. Digested tissues were centrifuged to remove any undigested parts, mixed with 0.5 ml isopropanol, and DNA was sedimented by centrifuging at 21000 g, for 8 minutes at 4° C. After one wash with 70% Ethanol, DNA was allowed to dry and was finally resuspended in 100 μl of 0.2×TE buffer (10 mM Tris, pH8.0, 1 mM EDTA).

### Example 5

Preparation of Plasmid DNA

Plasmid DNA was purified from overnight cultures, grown at 37° C. in LB containing Ampicilin (75 μg/ml), using Qiagen plasmid mini kits, QIAfilter plasmid midi, or maxi kits according to the manufacturer's instructions.

### Example 6

Ethanol Precipitation of DNA

DNA solutions were mixed with 0.1 volumes 8M LiCl and 3 volumes cold Ethanol. Glycogen was often added to facilitate visualization of the precipitate. The mixture was placed at −20° C. for 20 minutes; DNA was centrifuged at 21000 g for 20 minutes, washed twice with 70% Ethanol, air-dried and resuspended in TE buffer.

### Example 7

Quantitation of DNA

DNA solutions were placed in a quartz cuvette (1 cm path length) and the absorbance of the mixture was read at 260 nm and 280 nm using a Spectrophotometer. The OD₂₆₀ of 1, corresponding to 50 μg/ml double stranded or 33 μg/ml single stranded DNA, was used as a reference for DNA concentration calculations. The OD_{260/280} ratio was used as an estimate for purity of the preparation. A ratio of 1.6-1.9 was considered acceptable.

### Example 8

Restriction Enzyme Digestion

Restriction enzymes and buffers were purchased from New England Biolabs. For small scale, diagnostic, digests, 0.5 μg of DNA was incubated in the appropriate buffer with 5-10 U of restriction endonuclease in a total volume of 20 μl. The reaction was incubated at the appropriate temperature (25° C. or 37° C.) for 1-2 hours. For large scale digests, 5 μg of DNA were incubated in the appropriate buffer with 20-30 U of restriction endonuclease in a total volume of 70 μl. The reaction was incubated for 2-3 hours. Following digestion, enzymes were inactivated by incubating at 80° C. for 20 minutes.

### Example 9

Polymerase Chain Reaction (PCR)

Primers were 20-22 nucleotides in length, usually with two GC-basepairs at the 3′-end, and annealing temperatures of approximately 55° C. The annealing temperature specific to each primer was calculated using the following formula: Tm=3×(sum of GC-basepairs)+2×(sum of AT-basepairs). Reactions were carried in a peltier thermal cycler (MJ Research PCR Machine).

To genotype the TetO-VE-PTP strain, a combination of 3 primers were used in each reaction: TG primer recognized a sequence only present in the transgene, GE primer recognized a sequence only present in the endogenous copy of the gene, and CO primer recognized a region common to both the transgene and the endogenous versions of VE-PTP.

| | | |
|---|---|---|
| GE: | 5′-CTGCCACGGCCCTTGAGCATCG-3′ | (SEQ ID NO: 1) |
| TG: | 5′-CTCGGTACCCGGGTCGAGTAGGC-3′ | (SEQ ID NO: 2) |
| CO: | 5′-ACGCTCAGTGTTATCCACAAGGCCG-3′ | (SEQ ID NO: 3) |

2 μl of genomic DNA preparation from mouse tail were mixed with 40 pmols GE primer, 40 pmols TG primer, 80 pmols CO primer, and 15 μl Mega Mix Blue (Helena Biosciences). The reaction conditions were as follows:

| | | | |
|---|---|---|---|
| Segment 1 | 1 | cycle | 94° C. for 4 minutes |
| Segment 2 | 35 | cycles | 94° C. for 40 seconds |
| | | | 64° C. for 1 minute |
| | | | 72° C. for 1.5 minute |
| Segment 3 | 1 | cycle | 72° C. for 3 minutes |

Tie2tTa mice were genotyped using the following PCR primers:

| | |
|---|---|
| HHFW1: | |
| 5′-CGATACCATACATAGGTGGAGG-3′ | (SEQ ID NO: 4) |
| rtTAREV1: | |
| 5′-AATGGCTAAGGCGTCGAGCAAAG-3′ | (SEQ ID NO: 5) |

10 pmol of each primer were mixed with 2 μL tail DNA and 15 μl Mega Mix Blue (Helena Biosciences), and run according to the following PCR scheme:

| | | | |
|---|---|---|---|
| Segment 1 | 1 | cycle | 94° C. for 5 minutes |
| Segment 2 | 35 | cycles | 94° C. for 30 seconds |
| | | | 63° C. for 45 seconds |
| | | | 72° C. for 1 minute |
| Segment 3 | 1 | cycle | 72° C. for 8 minutes |

nZL2 and TRE-LacZ 2717 strains were genotyped using the following PCR primers:

| | |
|---|---|
| LacZFW4: | |
| 5′-CCGTCACGAGCATCATCCTC-3′ | (SEQ ID NO: 6) |
| LacZREV4: | |
| 5′-GACGAAACGCCTGCCAGTATTTAG-3′ | (SEQ ID NO: 7) |

13 pmol of each primer were mixed with 2 μl tail DNA and 15 μl Mega Mix Blue, and run using the same reaction conditions as in the Tie2-tTA PCR, only with an annealing temperature of 60° C.

### Example 10

DNA Sequencing

Primers of about 18 nucleotides were designed to amplify a region within the gene of interest or the vector sequence surrounding the gene. Reactions were carried out using 500 ng of a given plasmid, 3-4 pmol of primer, and 8 μl of fluorochrome labeling mix (CRUK), in a total volume of 20 μl. PCR conditions were the following:

| | | |
|---|---|---|
| Segment 1 | 25 cycles | denaturation at 96° C. for 10 seconds |
| | | annealing at 50° C. for 5 seconds |
| | | extension at 60° C. for 4 minutes |

Following amplification, DNA was ethanol precipitated, electrophoresed and visualized. SDS-PAGE electrophoresis and visualization were performed by CRUK Sequencing Service. Alternatively, DNA and appropriate primers were supplied to ACGT, Inc., to carry out the amplification and sequencing steps of the reaction. Sequence analysis and alignment was performed using the software programs MacVector (Accelrys) and Sequencher (Gene Codes Corporation).

The following oligonucleotides were used to sequence TetO-VE-PTP plasmids:

| | |
|---|---|
| Bluescript T3: | |
| 5′-AATTAACCCTCACTAAAGGG-3′ | (SEQ ID NO: 8) |
| Bluescript T7: | |
| 5′-GTAATACGACTCACTATAGGGC-3′ | (SEQ ID NO: 9) |
| Bluescript M13Fwd: | |
| 5′-GTAAAACGACGGCCAGT-3′ | (SEQ ID NO: 10) |
| VEP-A5: | |
| 5′-ACCTTCAGATTTGAGACTTCCATG-3′ | (SEQ ID NO: 11) |
| VEP-S3: | |
| 5′-CAGTGATACCAGCAGATACAGC-3′ | (SEQ ID NO: 12) |
| VEP-S4: | |
| 5′-GTGTCTGCATTCAGACAGGACG-3′ | (SEQ ID NO: 13) |
| VEP-S5: | |
| 5′-CTGTATGTGGTGACTCACAGTG-3′ | (SEQ ID NO: 14) |
| VEP-FW2: | |
| 5′-GGTTCAGCGACACCAACGGAG-3′ | (SEQ ID NO: 15) |
| VEP-FW4: | |
| 5′-CCTCTTGCCTGAGAATCGAGG-3 | (SEQ ID NO: 16) |
| Oligo(+): | |
| 5′-CGCGGCCGCTCTAGAGTTTAAACGGCGCGCC | (SEQ ID NO: 17) |
| TTAATTAAAGATCTG-3′ | |
| FWD-R/A: | |
| 5′-CATCCGTCACTTTCACTACAC-3′ | (SEQ ID NO: 18) |

All VEP-primers were provided by Dr. Urban Deutsch (Max-Planck Institute, Bad Nauheim, Germany)

The following oligonucleotides (Invitrogen) were used to sequence N-ezrin GFP and N-moesin GFP:

| | |
|---|---|
| T7 Sequencing Primer: | |
| 5′-TAATACGACTCACTATAGGG-3′ | (SEQ ID NO: 19) |
| GFP Reverse Primer: | |
| 5′-GGGTAAGCTTTCCGTATGTAGC-3′ | (SEQ ID NO: 20) |

### Example 11

DNA Agarose Gel Electrophoresis

Agarose gels were prepared by dissolving 0.6-2% agarose in TAE buffer (40 mM Tris Base, pH8.0, 20 mM glacial acetic acid, 1 mM EDTA). Ethidium bromide was added to a final concentration of 0.5 μg/ml. DNA was mixed with 10× BlueJuice™ gel loading buffer (Invitrogen) and electrophoresed at 5-20 V/cm in TAE buffer. A 100 bp or 1 kb DNA ladder (Invitrogen) was loaded in an adjacent well for comparison.

### Example 12

Gel Purification of DNA Fragments

Gel slices containing relevant DNA fragments were excised and stripped of agarose and contaminants using a gel extraction and nucleotide removal kit (Qiagen).

### Example 12

Cloning

Cohesive-End Ligation

Restriction digests for the insert and vector were performed. The vector fragment was also dephosphorylated with 0.5 U/μg DNA of alkaline phosphatase (CIP, New England Biolabs), for 1 hour at 37° C., to prevent recircularization in the case of compatible ends. Following purification, vector and insert were ligated using 200-300 ng total DNA with a 2-4 fold excess of insert, and 400 U T4 DNA ligase (New England Biolabs) in 15 μl total volume. The reaction was carried out overnight at 15° C. 1 μl of the ligation reaction was used to transform E. coli as described above.

Blunt-End Ligation

Restriction digests of vector and insert were performed as above. Fragments were incubated with T4 DNA polymerase (New England Biolabs) at 1-2 U/μg DNA, 200 μM dNTP (Ultrapure, Pharmacia), and 0.1 mg/ml BSA, at 12° C. for 20 minutes so as to form blunt ends. The polymerase was inactivated by incubating at 75° C. for 10 minutes, and the vector was dephosphorylated with alkaline phosphatase as above. Following purification, vector and insert were ligated as described above, this time using 2-fold excess of vector.

### Example 13

TOPO Cloning

The N-terminal domains of ezrin and moesin were cloned as GFP fusion products using the CT-GFP fusion TOPO® TA Expression kit (Invitrogen). N-ezrin (1-1191) and N-moesin (1-1143) were amplified from full-length clone CS0DF008YM15 (Invitrogen) and IMAGE clone 5044557 (Invitrogen), respectively. Taking into account the 3′-deoxyadenosine residues added by Taq polymerase to the PCR products, the following sets of primers were designed to ensure cloning in frame with the Cycle 3 GFP protein:

| | |
|---|---|
| Ezrin5′-primer: | |
| 5′-ACCGAAAATGCCGAAACC-3′ | (SEQ ID NO: 21) |
| Ezrin3′-primer: | |
| 5′-GCAGTGCAGCCATACGGTC-3′ | (SEQ ID NO: 22) |
| Moesin5′-primer: | |
| 5′-GCCACCATGCCGAAGACG-3′ | (SEQ ID NO: 23) |
| Moesin3′-primer: | |
| 5-GACGCTTCCGTTCCTGCTC-3′ | (SEQ ID NO: 24) |

1 μl of each primer was mixed with 100 ng of template DNA and 100 μl Platinum® PCR Supermix (Invitrogen) and PCR was carried out in a Peltier thermal cycler (MJ Research) as detailed below:

| | | | |
|---|---|---|---|
| Segment 1 | 1 | cycle | 95° C. for 1 minute |
| Segment 2 | 31 | cycles | 95° C. for 45 seconds |
| | | | 58° C. for 45 seconds |
| | | | 72° C. for 45 seconds |
| Segment 3 | 1 | cycle | 72° C. for 5 minutes |

2 μl of the 100 μl PCR reaction and 1 μl TOPO® vector were allowed to ligate in a final volume of 5 μl, for 5 minutes at room temperature. 2 μl of the ligation mixture was used to transform 50 μl of TOP10 Chemically competent cells as described above.

### Example 14

RNA Techniques

RNA Isolation

Total RNA was isolated from cells or tissues using the RNeasy mini kit (Qiagen). 3-5×10⁶ MEF cells were lysed in 350 μl RLT buffer (Qiagen) by pipetting, and the contents loaded on to 1 RNeasy mini column. 11.5 day old embryos were snap-frozen and immediately homogenized in 800 μl RLT buffer using a mortar and pestle. The lysate was passed through a QIAshredder spin column (Qiagen) and split between two RNeasy mini columns. RNA purification was carried out according to the manufacturer's instructions, with the additional step of on-column DNA digestion performed using the RNase-Free DNase Set (Qiagen). RNA was eluted in water and any remaining DNA was removed from the samples by treating 5 μg RNA with 1 U of rDNase I (DNA-free, Ambion) in a total volume of 50 μl for 1 hour at 37° C. rDNase I was inactivated by supplementing the reaction with DNase Inactivation reagent, and RNA was snap-frozen and stored at −80° C.

RNA Quantitation

RNA solutions were placed in a quartz cuvette (1 cm path length) and the absorbance of the mixture was read at 260 nm and 280 nm using a Spectrophotometer. The OD₂₆₀ of 1, corresponding to 40 μg/ml RNA, was used as a reference for calculations. To obtain an accurate OD_{260/280} ratio, RNA was diluted in 10 mM Tris HCl, pH 7.5. A ratio of 1.9-2.1 was expected for a pure preparation.

RNA Agarose Gel Electrophoresis

1.2% agarose gels were prepared in TBE buffer (45 mM Tris-borate pH 8.0, 1 mM EDTA) containing ethidium bromide at a final concentration of 0.5 μg/ml. RNA was mixed with 10× BlueJuice™ gel loading buffer (Invitrogen) and electrophoresed at 5-20 V/cm in TBE buffer. Sharp and distinct ribosomal RNA bands, with the 28S ribosomal RNA band of double intensity to the 18S RNA band, were an indication of intact RNA.

RT-PCR

Reverse transcription of RNA was performed using 1 μg RNA, and a two-step reaction using Superscript™ Rnase H⁻ Reverse Trasncriptase (Invitrogen): RNA, 1 μl Oligo (dT)₁₂₋₁₈ (Invitrogen), 1 μl 10 mM dNTP (Invitrogen), in a total volume of 12 μl, were heated to 65° C. for 5 minutes in a thermal cycler. After cooling on ice, the reaction was supplemented with 4 μl 5× First-Strand buffer, 2 μl 0.1 M DTT, 1 μl RNaseOUT™ (Invitrogen), and heated to 42° C. for 2 minutes. Finally, 1 μl SuperScript™ II was added, and the reaction was incubated at 42° C. for 50 minutes. The enzyme was inactivated by heating for 15 minutes at 70° C.

To specifically look at transgene VE-PTP, primers were designed to amplify a region extending to the transcribed exogenous promoter sequence and also absent from the endogenous copy of the gene:

| | | |
|---|---|---|
| rt-tg3: | ATGCACTAGTGGCGCCTGTCG | (SEQ ID NO: 25) |
| rt-rev1: | ACTTGCTCTGCCACTCCAGTCTGC | (SEQ ID NO: 26) |

The housekeeping gene p97 was selected as an internal control, and was amplified with primers that spanned an intron, so as to distinguish between cDNA and any genomic DNA contaminant:

| | |
|---|---|
| p97.RT.1859S: | |
| 5′-ACGGCAAGCTGCCCCCTGTG-3′ | (SEQ ID NO: 27) |
| p97.RT.2321A: | |
| 5′-CATAGCCGATGGATTTGTCTGC-3′ | (SEQ ID NO: 28) |

For each PCR reaction, 2 μl cDNA was mixed with 15 μl Mega Mix Blue (Helena Biosciences), 40 pmol of each transgene VE-PTP primer, and 1 pmol of each p97 primer. The reaction conditions were the same as for genotyping TetO-VE-PTP mice.

### Example 15

Real Time Quantitative PCR (Taqman)

Reverse transcription of RNA was performed using 300 ng RNA and the following reaction mix (ABI): 1×RT buffer, 5.5 mM MgCl₂, 500 μM of each dNTP, 2.5 μM random primers, 0.4 U/μl Rnase Inhibitor, 1.25 U/μl Multiscribe Reverse Transcriptase, supplemented with Nuclease-Free water (Ambion) to a total volume of 60 μl. cDNA synthesis was carried out in a thermal cycler according to the following scheme:

| | | |
|---|---|---|
| Segment 1 | 1 cycle | 25° C. for 10 minutes |
| Segment 2 | 1 cycle | 42° C. for 60 minutes |
| Segment 3 | 1 cycle | 95° C. for 5 minutes |

Taqman cocktails were prepared using 1×PCR master mix (ABI), 5 μl cDNA, 250 nM of each primer, and 500 nM of probe, in a total volume of 25 μl. The following primer/probe sets were used:

| | |
|---|---|
| Endogenous VE_PTP Fwd: | |
| 5′-CCA CCG GCC CTT GAG CAT-3′ | (SEQ ID NO: 29) |
| Endogenous VE_PTP Rev: | |
| 5′-CCA GCC AGG GCA CTT CTG-3′ | (SEQ ID NO: 30) |
| Endogenous VE_PTP Probe: | |
| 5′-CGC TCA ACA AGT GGT AC-3′ | (SEQ ID NO: 31) |
| Transgene VE_PTP Fwd: | |
| 5′-GCA TGC AAG CTT CAC ATA TGC-3′ | (SEQ ID NO: 32) |
| Transgene VE_PTP Rev: | |
| 5′-CCA GCC AGG GCA CTT CTG-3′ | (SEQ ID NO: 33) |
| Transgene VE_PTP Probe: | |
| 5′-CGC TCA ACA AGT GGT AC-3′ | (SEQ ID NO: 34) |
| Total VE_PTP Fwd: | |
| 5′-TAA CAC TGA GCG TCG TGC AGA-3′ | (SEQ ID NO: 35) |
| Total VE_PTP Rev: | |
| 5′-CTA GAG ACC CTG GAC TCC AAC | (SEQ ID NO: 36) |
| AG-3′ | |
| Total VE_PTP Probe: | |
| 5′-CAG AGC AAG TGA AAT GTA-3′ | (SEQ ID NO: 37) |
| Tie2-tTA Fwd: | |
| 5′-ACG GCG CTC TGG ATA TGG-3′ | (SEQ ID NO: 38) |
| Tie2-tTA Rev: | |
| 5′-TTC CAA GGG CAT CGG TAA AC-3′ | (SEQ ID NO: 39) |
| Tie2-tTA Probe: | |
| 5′-CGA CTT CGA GTT TGA GCA-3′ | (SEQ ID NO: 40) |

For VE-PTP, endogenous primer/probe sets were designed to only amplify the endogenous VE-PTP copy, transgene primer/probe sets to only amplify the transgene copy, while total VE-PTP primer/probe sets were non-discriminatory between the two versions.

GAPDH served as the internal control in all reactions, and was amplified with rodent GAPDH primers and probe (ABI).

Reactions were loaded on Prism Optical tubes (ABI) and run in the ABI Prism 7700 Sequence Detection System using the following cycling conditions:

| | | | |
|---|---|---|---|
| Segment 1 | 1 | cycle | 50° C. for 2 minutes |
| Segment 2 | 1 | cycle | 95° C. for 10 minutes |
| Segment 3 | 40 | cycles | 95° C. for 15 seconds |
| | | | 60° C. for 1 minute |

Results were analysed using the SDS 7900HT Software 2.2 (ABI).

### Example 16

Generation of Transgenic Mice

Construction of Transgene Constructs for TetO-VE-PTP

The following steps were taken to generate the vector that would harbour the responder transgene constructs:
1) plasmid pRL-null (Promega) was digested with NheI and NotI to remove the luciferase gene, blunted and religated to produce pSI1.
2) An XhoI/BamHI fragment of pSI1, containing a multiple cloning site, an artificial intron, and an SV40 late poly (A) signal, was cloned into XhoI/BamHI-digested pBluescript® II KS (Stratagene) to produce pSI2.
3) The operator sequences from pUHC 13-3 [1] were removed by an XhoI/SalI double digest and cloned into Xho/SalI-digested pSI2 to produce pSI3 (Gossen, M. and H. Bujard, Tight control of gene expression in mammalian cells by tetracycline-responsive promoters. Proc Natl Acad Sci USA, 1992. 89(12): p. 5547-51)
4) An oligo containing a series of rare restriction sites (GGGCCCGCGGCCGCTCTAGAGTTTAAACGGCGCGCCTTAATTAAAGA TCTGTCGAC) (SEQ ID NO: 41) was digested with Apa/SalI and cloned into ApaI/XhoI-digested pSI3 to produce pSI4.

Full-length VE-PTP was excised from pCMV6-XL4 (Genebank AF067196) using Not I, and cloned into the multiple cloning site of pSE420 (Invitrogen), in reverse orientation with respect to the vector open reading frame. VE-PTP cDNA was released from the resulting vector by a BssHII digest, and cloned into MluI-digested pSI4, to yield pSI5, a plasmid harbouring the cDNA of VE-PTP under the control of the tetracycline response element.

To construct a plasmid with only the extracellular domain of VE-PTP expressed as a soluble protein under the control of the tetracycline response element, the following steps were taken: A 300 bp fragment from an AccI/XbaI digest of pFLAG-CMV-1-V-sol FN17 (provided by Dr. Urban Deutsch), was ligated to the 8 kb fragment from an AccI/XbaI digest of pCMV6-XL4 VE-PTP FN17Fc (provided by Dr. Urban Deutsch). The resulting plasmid was cut with Pml I and NheI, and the 3.2 kb fragment was ligated to the 5.5 kb fragment of pSI5 digested with Pml I and XbaI, to yield pSI9.

To construct a plasmid with the phosphatase trapping mutant of VE-PTP under the control of the tetracycline response element, pCMV-FLAG1 VE-PTP R/A (provided by Dr. Urban Deutsch) (Fachinger, G., U. Deutsch, and W. Risau, Functional interaction of vascular endothelial-protein-tyrosine phosphatase with the angiopoietin receptor Tie-2. Oncogene, 1999. 18(43): p. 5948-53) was digested with Nhe I/Xba I and the resulting 1.3 kb fragment was ligated to the 9 kb fragment of the NheI-digested pSI5 to yield pSI10.

Injection of TetO-VE-PTP Transgenes into Mouse Oocytes

Transgenes TetO-VE-PTP, TetO-VE-PTP EC, and TetO-VE-PTP R/A were linearized by digesting 50 μg of the respective plasmids pSI5, pSI9, and pSI10, with BssHII. The 6-7 kb transgenes were separated from the vector on a 1% Agarose Gel in TBE without ethidium bromide. The marker lane and part of the lane containing the digestion products were cut and stained in a solution of 0.5 μg/ml Ethidium Bromide in TBE for 30 minutes, to avoid exposure of the DNA to UV light. Using the position of the transgene in the stained gel as a landmark, the relevant portion of the unstained gel was excised and gel-purified. Eluates were ethanol precipitated and resuspended in EB buffer (10 mM Tris HCl, pH 8.5). A further round of purification was achieved by diluting the DNA in 2.4 ml of 10 mM Tris HCl, pH 8.0, 1 mM EDTA, adding 3.0 g of ultrapure CsCl, and ultracentrifuging in a SW50.1 rotor at 20° C. for 48 hours at 40,000 rpm. Fractions containing the DNA were pooled and dialyzed over 48-hours at 4° C., against a large volume of injection buffer. The solution was filtered through a 0.2 μm filter, and adjusted to a concentration of 1 ng/μl.

0.5 day old mouse embryos were collected from the oviducts of superovulated and mated C57BL/6 or (C57BL/6×CBA) female mice. Using a glass holding pipette between 80 and 120 μm in diameter, 1-2 μl of the DNA solution was microinjected into the pronuclei of the one-cell embryos prior to the 1^{st} division. Once microinjected, healthy eggs were transferred into the oviducts of 0.5 day pseudopregnant surrogate female mice. Approximately 20-30 microinjected eggs were transferred into each pseudopregnant recipient.

Breeding and Maintaining of Mouse Colony

Transgenic mice were maintained in C57BL/6 or mixed (C57BL/6×CBA) background. Experimental crosses were performed with Tie2tTA, nZL2, TRE-LacZ 2717 strains (provided by Dr. Urban Deutsch).

For timed-matings, the morning of vaginal plug formation was counted as 0.5 dpc. Tails were removed for genotyping pups and adult mice, and yolksacs were used to genotype embryos as described above.

### INCORPORATION BY REFERENCE

The patent and scientific literature referred to herein establishes knowledge that is available to those of skill in the art. All issued patents, patent applications, published foreign applications, and published references, including GenBank database sequences, which are cited herein, are hereby incorporated by reference to the same extent as if each was specifically and individually indicated to be incorporated by reference in their entirety.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

We claim:

1. A double transgenic mouse capable of conditionally over-expressing VE-PTP which is contingent on the presence of doxycycline.

2. The double transgenic mouse of claim 2, wherein the transgenic mouse is C57BL/6 or C57BL/6×CBA.

3. A transgenic responder line mouse whose genome comprises a TetO-VE-PTP transgene.

4. The transgenic responder line mouse of claim 3, wherein the transgenic responder line mouse is C57BL/6 or C57BL/6×CBA.

5. A method of producing a transgenic responder line mouse whose genome comprises a TetO-VE-PTP transgene comprising the steps of:
a) introducing a DNA solution comprising TetO-VE-PTP into mouse oocytes;
b) transferring the mouse oocytes to a pseudo-pregnant surrogate female mouse; and
c) allowing the oocytes to develop to term thereby producing a transgenic responder line mouse whose genome comprises a TetO-VE-PTP transgene.

6. The method of claim 5, wherein the transgenic responder line mouse is C57BL/6 or C57BL/6×CBA.

7. A double transgenic mouse whose genome comprises the transgenes TetO-VE-PTP and Tie2-tTA.

8. The double transgenic mouse of claim 7, wherein the double transgenic mouse is C57BL/6 or C57BL/6×CBA.

9. A method of producing a double transgenic mouse comprising: crossing a transgenic mouse responder line comprising TetO-VE-PTP with a transgenic mouse driver line comprising Tie2-tTA.

10. The method of claim 9, wherein the double transgenic mouse is C57BL/6 or C57BL/6×CBA.
